(19) United States
(12) Patent Application Publication
(10) Pub. No.: US 2020/0405533 A1
(43) Pub. Date: Dec. 31, 2020

(54) Wearable Therapy System

(71) Applicant: In-Covery Systems, Inc., Prescott, AZ (US)

(72) Inventors: James R. Check, Prescott, AZ (US); Heather S. Royer, Prescott, AZ (US)

(73) Assignee: In-Covery Systems, Inc., Prescott, AZ (US)

(21) Appl. No.: 16/929,255

(22) Filed: Jul. 15, 2020

Related U.S. Application Data
(63) Continuation-in-part of application No. 16/428,237, filed on May 31, 2019.

(51) Int. Cl. A61F 7/02; A61F 7/10

(52) U.S. Cl. CPC A61F 7/02; A61F 2007/0056; A61F 7/10

(57) ABSTRACT

A wearable therapy system comprises a therapy pack, a therapy pack holder including a body and a first fastener system, the body being configured to removably receive the therapy pack, and a therapy garment configured to be worn by a user and comprising at least one attachment area including a second fastener system. The therapy pack holder is removably attachable to the attachment area in a plurality of positions with the first fastener system engaging the second fastener system to secure the therapy pack in relation to the therapy garment. The system of the present disclosure can also include one or more compression sleeves and/or therapeutic wraps configured to be worn by the user that comprise fastener systems configured to removably accept one or more of the therapy packs, pads, and holders in a plurality of positions relative to the one or more compression sleeves.

### CROSS-REFERENCE TO RELATED APPLICATION

This application is a continuation-in-part application of, and claims priority to, U.S. patent application Ser. No. 16/428,237, filed on May 31,2019, the entire contents of which are hereby expressly incorporated by reference.

### BACKGROUND

### Field of the Disclosure

The present disclosure relates generally to a wearable therapy system. More specifically, the present disclosure relates to a wearable therapy system comprising wearable articles that can be secured about a user and therapeutic hot and cold packs capable of being attached to and detached from the wearable articles.

### Related Art

Heat therapy and cold therapy are commonly used to alleviate pain, swelling, inflammation, and other conditions related to surgery, injury, exercise, and natural conditions. Heat therapy improves circulation and blood flow to a particular area. Specifically, increasing the temperature of an afflicted area can soothe discomfort, increase muscle flexibility, and heal damaged tissue. Cold therapy reduces blood flow to a particular area, which can reduce inflammation and swelling. Additionally, cold therapy reduces nerve activity, which can relieve pain.

To apply heat or cold therapy, therapeutic pads (e.g., ice/heat packs) are commonly used on the afflicted area. Generally, a person holds the ice/heat pack in a desired position against the body for a time period, or attaches the ice/heat pack to the body via adhesive tape or an elastic bandage wrap. Such methods are cumbersome, irritating, and can be difficult for a single person to employ. In addition, such methods are hard to apply when the person is injured and can become loose or undone during movement. In view of the foregoing, it is desirable to provide a wearable therapy system capable of providing hot and cold therapies with convenience and ease.

### SUMMARY

The present disclosure generally relates to a wearable therapy system for an individual. Specifically, the wearable therapy system comprises a therapy pack, a therapy pack holder including a body and a first fastener system, the body being configured to be worn by a user and to removably receive the therapy pack, and a therapy garment comprising at least one attachment area including a second fastener system. The therapy pack holder is removably attachable to the attachment area in a plurality of positions with the first fastener system engaging the second fastener system to secure the therapy pack in relation to the therapy garment.

The wearable therapy system of the present disclosure can also include a compression sleeve that is configured to be worn around a user's limb. The compression sleeve can have an exterior layer and an interior layer. The interior layer can be formed of a stretchable material and include a fastener system, and the interior layer can be configured to exert a compressive force around the user's limb. The therapy pack holder can be removably attached to the exterior layer of the compression sleeve in a plurality of positions in relation thereto. In one aspect, the stretchable material of the exterior layer can be a four-way stretchable material, and the fastener system of the therapy pack holder can be configured to engage the stretchable material to removably attach the therapy pack holder to the compression sleeve. In some aspects, the interior layer can be formed from an elastomeric material. According to additional aspects of the present disclosure, a plurality of compression sleeves of various configurations can be stacked together on the user's limb, forming a compression sleeve support system.

The wearable therapy system of the present disclosure can also include a therapy pad holder and a therapy pad that is configured to have fluid circulated threthrough. The therapy pad holder can comprise a pouch with an opening and a fastener system. The opening can be configured to removably receive the therapy pad, and the pouch can be configured to conform to the geometry of the therapy pad and prevent movement thereof. The fastener system of the therapy pad holder is removably engageable with a fastener system of a therapeutic garment that is configured to be worn about a user's body, such that the therapy pack can be removably attached to the therapeutic device in a plurality of positions in relation thereto. The pouch of the therapy pad holder can be formed from a stretchable netting. The therapy pad holder can be configured conform to therapy pads having irregular configurations and dimensions.

The wearable therapy system of the present disclosure can also include a therapeutic wrap that is configured to be wrapped about at least a portion of a user's body. The therapeutic wrap can include a first fastener system on a first side thereof and a second fastener system on a second side thereof that is removably engageable with the first fastener system on the first side of the therapeutic wrap. As such, the therapeutic wrap can be wrapped and secured around at least a portion of the user's body. The first fastener system can comprise a hook an loop fastener system that can be stretched in one or more directions. A fastener system of a therapy pack and/or therapy pad can be removably engageable with the first fastener system, such that the therapy pack and/or therapy pad can be removably attached to the therapeutic wrap in a plurality of positions in relation thereto. In some aspects, the first fastener system can be a four-way stretchable material. In such aspects, the fastener system of the therapy pack and/or therapy pad can be configured to engage the stretchable material to removably attach the therapy pack and/or therapy pad to the therapeutic wrap, and the second fastener system of the therapeutic wrap can be configured to engage the stretchable material to removably secure the therapeutic wrap around at least a portion of the user's body. In other aspects, the therapeutic wrap can include a first layer formed of the stretchable material and a second layer formed of an elastomeric material.

### BRIEF DESCRIPTION OF THE DRAWINGS

The foregoing features of the disclosure will be apparent from the following Detailed Description, taken in connection with the accompanying drawings, in which:

FIG. 1 is an illustration showing a therapy system of the present disclosure including a therapy garment, a therapy pack, and a therapy pack holder;

FIG. 2 is an illustration showing the therapy pack of the therapy system of FIG. 1 in greater detail;

FIG. 3A is a front view of the therapy pack holder of the therapy system of FIG. 1 in greater detail;

FIG. 3B is a rear view of the therapy pack holder of the therapy system of FIG. 1 in greater detail;

FIG. 4A is a front view of the therapy garment of the therapy system of FIG. 1 in greater detail;

FIG. 4B is a rear view of the therapy garment of the therapy system of FIG. 1 in greater detail;

FIG. 5 is a perspective view of a compression sleeve of the therapy system of the present disclosure;

FIG. 6 is an exploded view illustrating components of a stackable compression and support therapy system of the present disclosure;

FIG. 7 is a front view of a therapy pad holder of the therapy system of the present disclosure; and

FIG. 8 is a front view of a therapeutic wrap of the therapy system of the present disclosure.

### DETAILED DESCRIPTION

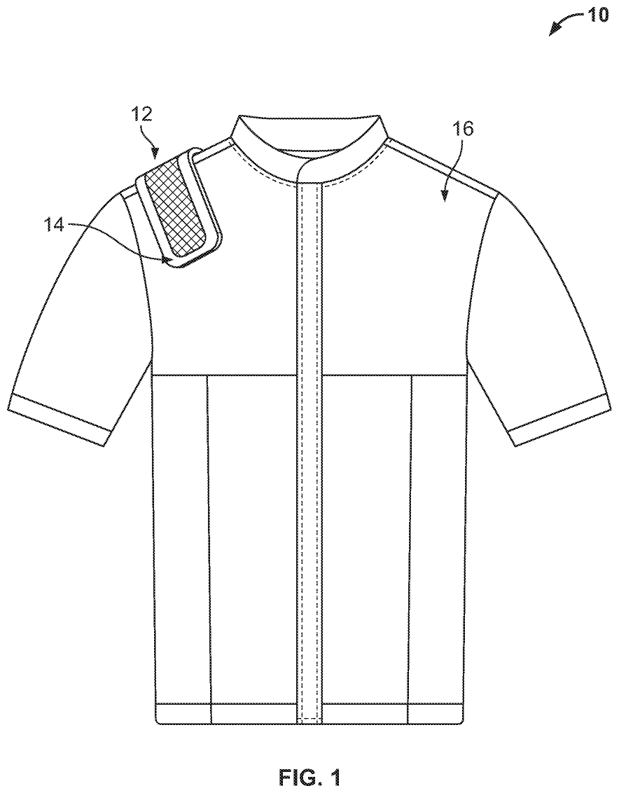

The present disclosure relates to a therapy system comprising wearable articles that can be secured about a user's body and therapy packs and pads capable of being removably attached thereto, as described in detail below in connection with FIGS. 1-8.

FIG. 1 is an illustration showing the therapy system 10 of the present disclosure. The therapy system 10 comprises a therapy pack 12, a therapy pack holder 14, and a therapy garment 16. The therapy pack 12 can be, for example, a hot or cold pack/pad, and can be removably housed in the therapy pack holder 14. The therapy pack holder 14 can be attached to different locations on the therapy garment 16, and detached therefrom as desired. This allows the therapy pack 12 to provide therapy, e.g., hot or cold therapy, to a targeted location on a user. For example, the user (after a workout) can attach a cold therapy pack to their shoulder to alleviate pain, and a heated therapy pack to their chest to improve circulation and soothe discomfort.

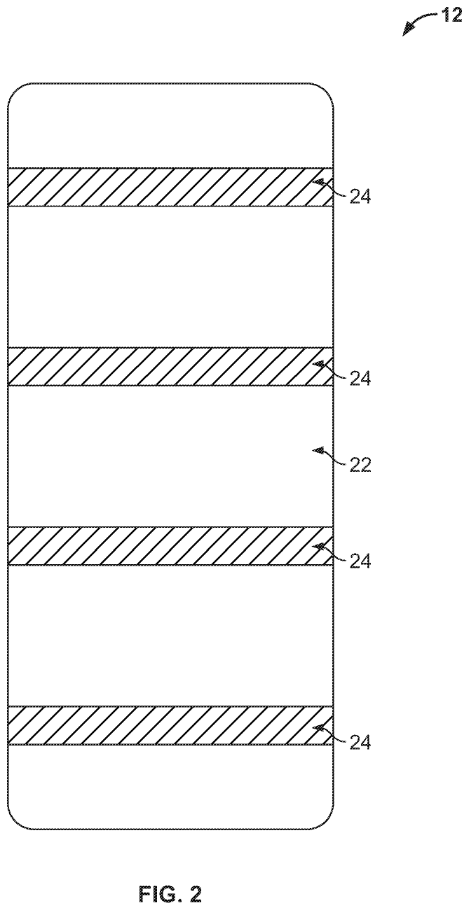

FIG. 2 is an illustration of the therapy pack 12. The therapy pack 12 comprises a flexible pouch 22 filled with a material capable of retaining and/or generating heat or cold, such as a hot pack or a cold pack, and one or more securing strips 24. The therapy pack 12 can be a reusable pack or a single use pack. The material inside the therapy pack 12 can comprise water, a refrigerant gel, any other liquid, ammonium nitrate, calcium ammonium nitrate, urea, hydroxyethyl cellulose, sodium polyacrylate, vinyl-coated silica gel, sodium acetate, iron powder, grains, or any other material/combination of materials capable of retaining or producing cold or heat.

The securing strips 24 enable the therapy pack 12 to be stably positioned within the therapy pack holder 14. The securing strips 24 can comprise any material with adhesive, fastening, or gripping properties, such as, but not limited to, silicone, a hook and loop fastener system (e.g., Velcro®), adhesive tape, rubber, etc. It should be understood that any amount of securing strips 24 can be attached to the pouch 22, and in any configuration.

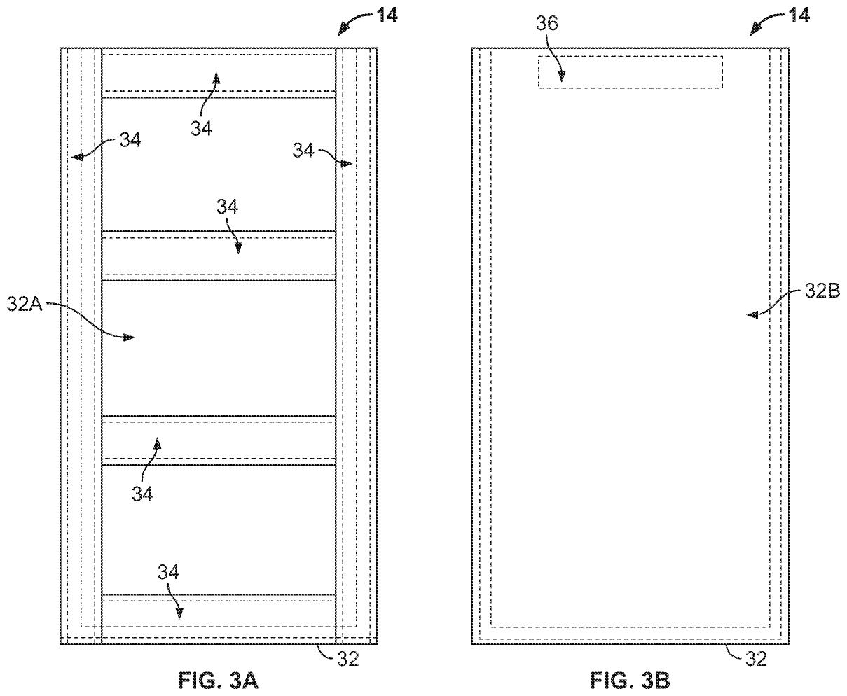

FIGS. 3A and 3B are first and second illustrations showing the therapy pack holder 14 of the therapy system 10. Specifically, FIG. 3A is a front view of the therapy pack holder 14 and FIG. 3B is a rear view of the therapy pack holder 14. The therapy pack holder 14 comprises a pouch 32 having a front side 32A and a back side 32A, a fastener system 34, and a closure mechanism 36. The pouch 32 can be formed of various materials, such as, a rubber material (e.g., neoprene, latex, other synthetic or natural rubbers), a cloth material, a synthetic material, a natural material, a material with dry wick capabilities, any other suitable material as known by those skilled in the art, or any combination of the foregoing materials. Additionally, the pouch 32 can be formed in various configurations, including but not limited to, a solid configuration, a mesh-type configuration, a netting-type configuration, a weave configuration, any other suitable combination as known by those skilled in the art, or any combination of the foregoing configurations. By way of example, the front side 32A of the pouch can comprise a first material in a solid configuration while the back side 32A of the pouch can comprise a second material in a mesh configuration.

The fastener system 34 is attached to the pouch 32 and can comprise any type of system capable of removably attaching the therapy pack holder 14 to the therapy garment 16. By way of example, the fastener system 34 discussed herein could be a hook and loop fastener system (or one component thereof), such as Velcro®. However, those skilled in the art would understand that any other suitable fastener system can be used for attaching and detaching the therapy pack holder 14 to the therapy garment 16, such as but not limited to, an adhesive system, a magnetic system, a zipper system, a button system, a clip system, etc. In another example, the therapy pack 12 itself can include a fastener system such that is can be attached directly to the therapy garment 16.

The hook and loop fastener system comprises a hook component having small hooks, and a loop component having small loops. Pressing the hook component to the loop component causes the hooks to attach to the loops and fasten the hook component to the loop component. By way of example, the fastener system 34 can comprise a hook component, and can attach to a loop component of the therapy garment 16 (or vice versa).

The pouch 32 can comprise one or more openings. The therapy pack 12 can be inserted into the pouch 32 via the one or more openings. The closure mechanism 36 is used to aid in securing the therapy pack 12 inside the pouch 32. For example, the closure mechanism 36 can comprise a hook and loop fastener system inside the pouch 32. Specifically, a hook component can be attached to an interior top edge of the back side 32B of the of the pouch and a loop component can be attached to an interior top edge of the front side 32A of the pouch. Alternatively, the closure mechanism 36 can comprise a flap system, an adhesive system, a zipper system, a button system, a magnetic system, a clip system, or any other suitable system to retain the therapy pack 12 inside the pouch 32.

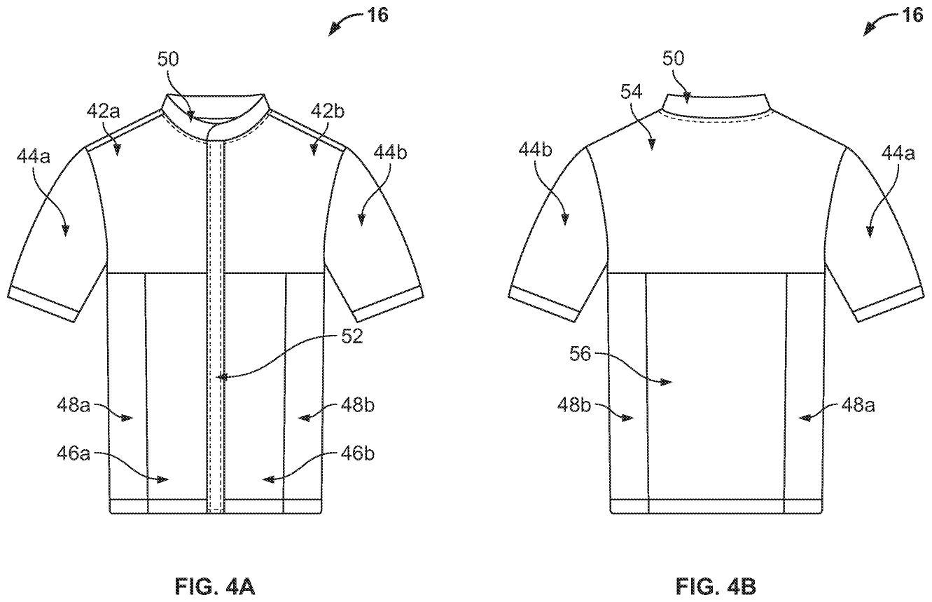

FIGS. 4A and 4B are illustrations showing the therapy garment 16 of the therapy system 10 in greater detail. Specifically, FIG. 4A is a front view of the therapy garment 16 and FIG. 4B is a back view of the therapy garment 16. The therapy garment 16 can comprise a plurality of sections, including a first front yoke 42a, a second front yoke 42b, a first sleeve 44a, a second sleeve 44b, a first midsection 46a, a second midsection 46b, a first side section 48a, a second side section 48b, a collar 50, a placket 52, a back yoke 54, and a back section 56. The entirety, or discrete portions of each section of the therapy garment 16 can comprise a fastener system 34 capable of having the therapy pack holder 14 removably attached thereto. By way of example, the first front yoke 42a, the second front yoke 42b, the first sleeve, 44a, the second sleeve 42b, and the back section 56 can comprise a loop component, to which a hook component of the therapy pack holder 14 can be attached. As such, the therapy pack holder 14 can be removably attached to any portion of the first front yoke 42a, the second front yoke 42b, the first sleeve, 44a, the second sleeve 42b, and the back section 56. However, it is understood that the garment sections can comprise any attachment system discussed herein. Further, it is noted that the garment section discussed is for illustrative purposes and that any combination of garment sections can be combined to form a single section or different sections.

Moreover, any combination of the foregoing sections can include a fastener system. It is noted that although entire sections are described as being formed of a fastener material, it should be understood that individual strips can be placed in various locations on one or more of the sections.

The collar 50 can be raised and comprise of a padded or soft material. This allows the collar 50 to lessen discomfort associated with the user using a sling (e.g., due to surgery, injury, etc.), placing the therapy pack holder 14 near the neck area, etc. The placket 52 can comprise an attachment system to close the therapy garment 16 onto the user. For example, the placket 52 can comprise a hook and loop fastener system, hook and loop buttons, a zipper system, a button system, etc.

As discussed above, the therapy system 10 allows the user to wear the therapy garment 16 and removably attach one or more therapy packs 12, either alone or held by a therapy holder 14, to the therapy garment 16. It is noted that the use of a shirt as the therapy garment 16 is by way of example only, and that other garments can be used as the therapy garment 16, including but not limited to, long sleeve shirts, compression garments, sweatshirts, hooded sweatshirts, jackets, pants, shorts, gloves, braces (e.g., shoulder braces, elbow braces, wrist braces, knee braces, ankle braces, etc.) hats, masks, etc.

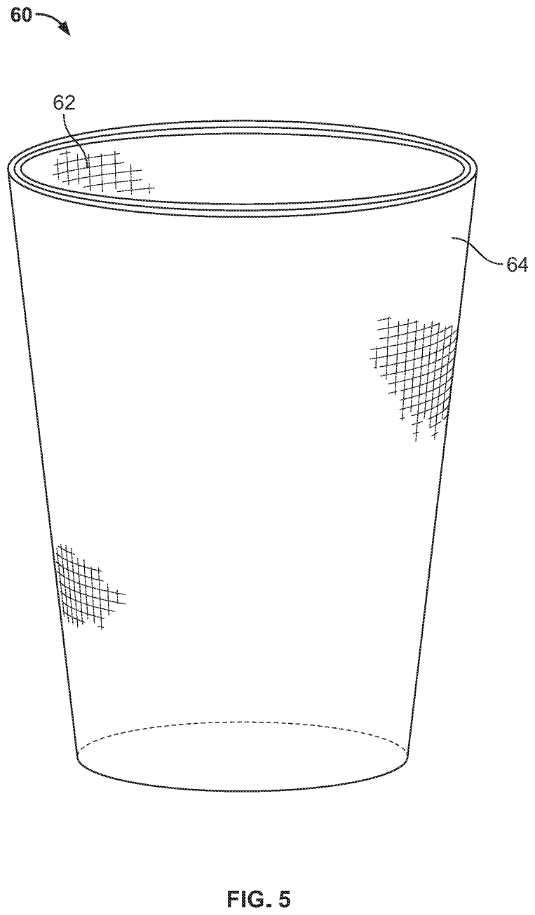

For example, FIG. 5 is a perspective view of a compression sleeve 60 that can be used in connection with the therapy system 10 of the present disclosure. As shown, the compression sleeve 60 can be formed from a plurality of layers, including but not limited to, an interior layer 62 and an exterior layer 64. The interior layer 62 can be formed from an elastomeric material that is configured to exert a compressive force on or around a user's limb (e.g., knee, elbow, calf, etc.), thereby securing the compression sleeve 60 to the limb and providing support thereto. At least a portion of the exterior layer 64 of the compression sleeve 60 can comprise a fastening system, such as fastening system 34, that is capable of removably securing one or more therapy pack holders 14, or therapy packs 12, to the exterior surface of the compression sleeve. For example, at least a portion of the exterior layer 64 can be formed from a material that is capable of engaging the securing strips 24 of the therapy pack 12 or the fastening system 34 of the therapy pack holder 14, e.g., a loop-type material. In this regard, the exterior layer 64 can be formed from a four-way stretch material, e.g., a material that can be stretched in one or more directions (e.g., lateral and/or transverse) so that the exterior layer 64 can stretch and/or move in unison with the interior layer 62, which can be compatible with the securing strips 24 of the therapy pack 12 or the fastening system 34 of the therapy pack holder 14. That is, the securing strips 24 and fastening system 34 can engage the material of the exterior layer 64 to secure the therapy pack 12 or the therapy pack holder 14 to the compression sleeve 60. Additionally, such four-way stretch materials can be configured to produce minimal to no fabric pilling when engaged by a hook-type fastener.

According to some aspects of the present disclosure, the interior layer 62 and the exterior layer 64 can be sewn together, however, any mechanism for securing the layers together can be utilized. According to still further aspects of the present disclosure, the interior layer 62 and the exterior layer 64 can be integrally formed. As such, the compression sleeve 60 can be formed from a single layer of elastomeric hook and loop material that provides both compressive support, as well as the capability to removably secure one or more therapy pack holders 14 or therapy packs 12 to the exterior surface 64 of the compression sleeve.

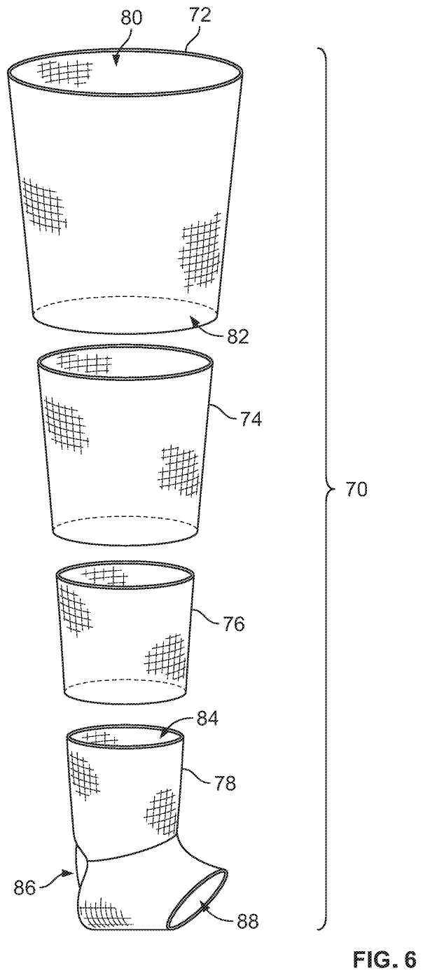

FIG. 6 is an exploded view of a stackable compression support system 70 according to the present disclosure. As shown, the compression support system 70 can include, but is not limited to, a thigh compression sleeve 72, a knee compression sleeve 74, a calf compression sleeve 76, and an ankle/foot compression sleeve 78. The compression sleeves 72, 74, 76, 78 can be used separately (e.g., to support a particular portion of a limb) or the compression sleeves 72, 74, 76, 78 can be used together (e.g., forming a compression sleeve that provides support to the entire limb). Ease of use of the compression support system is enhanced for patients, who potentially have limited strength and mobility, because the compression sleeves 72, 74, 76, 78 can be worn individually, which allows for targeted therapy. Mobility (e.g., bending) at the knee and/or elbow is also enhanced because the compression sleeves can move relative to each other.

Each of the compression sleeves 72, 74, 76, 78 can have a generally similar construction as the compression sleeve 60 shown and described in connection with FIG. 5. That is, the compression sleeves 72, 74, 76, 78 can have an interior layer formed from an elastomeric material and an exterior layer that includes a fastening system or is otherwise compatible with the securing strips 24 of the therapy pack 12 or the fastening system 34 of the therapy pack holder 14. For example, the exterior layer of the compression sleeves 72, 74, 76, 78 can be formed of a hook and loop material (or any of the other fastening systems disclosed herein) that can be stretched in one or more directions, e.g., a four-way stretch material for securing one or more therapy pack holders 14 or therapy packs 12 to the exterior surfaces of the compression sleeves 72, 74, 76, 78.

Each of the compression sleeves 72, 74, 76, 78 can be sized and configured for use with one or more body parts (e.g., thigh, knee, calf, ankle/foot, and the like). For example, the thigh compression sleeve 72 can have a generally cylindrical shape that tapers from a larger upper opening 80 to a smaller lower opening 82 (e.g., to accommodate the musculature of a user's thigh), whereas the ankle/foot compression sleeve 78 can have a generally “L” shaped construction with an upper opening 84, a rear opening 86 to accommodate a user's heel, and a front opening 88 to accommodate a user's toes. Additionally, each of the compression sleeves 72, 74, 76, 78 can be provided in a plurality of sizes (e.g., small, medium, large, extra large, etc.) in order to accommodate users of various sizes. Additional compression sleeves (not shown) can also be provided that are sized and configured to form a compression support system for a user's arm. For example, the compression support system for the user's arm can include a biceps compression sleeve, a biceps/elbow/forearm compression sleeve, and a wrist/hand/thumb compressions sleeve. Of course, appropriately sized compression sleeves from the compression support system 70 can be used to provide support for one or more portions of a user's arm. For example, a small knee compression sleeve 74 can be used to provide support to a user's biceps, a small calf compression sleeve 76 can be used to support a user's forearm or wrist, and a small ankle/foot compression sleeve can be used to provide support to a user's elbow. Accordingly, the compression support system 70 can include one or more of compression sleeves 72, 74, 76, 78 provided in various sizes, which can be used to support a user's leg and/or arm based on the specific needs of the user.

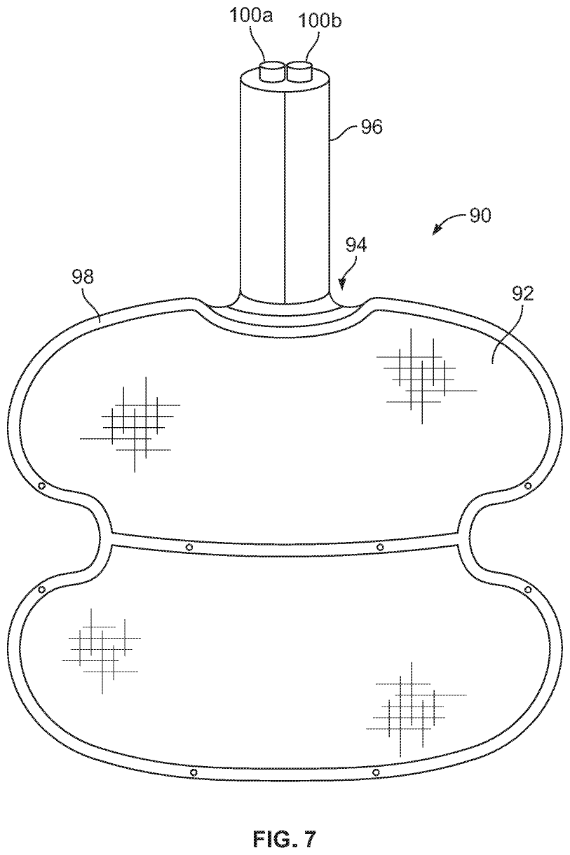

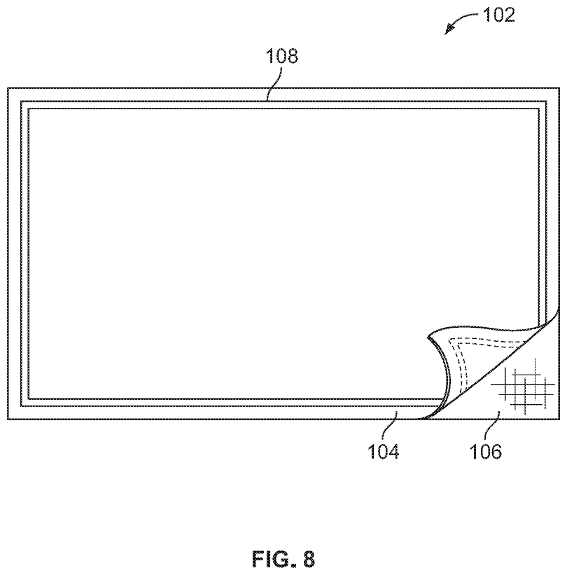

FIG. 7 is a front view of a therapy pad holder 90 according to aspects of the present disclosure. The therapy pad holder 90 can include a pouch 92 for accepting and securing a therapy pad 96, and a fastener system 98 for securing the therapy pad holder 90 to one or more of the garment 16, the sleeve 60, one or more of the sleeves 72, 74, 76, 78 of the compression support system 70, or a therapeutic wrap 102 (see, e.g., FIG. 8). The pouch 92 includes one or more openings 94 and can be formed of various materials, such as, a rubber material (e.g., neoprene, latex, other synthetic or natural rubbers), a cloth material, a synthetic material, a natural material, a material with dry wick capabilities, or any other suitable material as known by those skilled in the art, as well as any combination of the foregoing materials. Additionally, the pouch 92 can be formed in various configurations, including, but not limited to, a solid configuration, a mesh-type configuration, a netting-type configuration, a weave configuration, or any other suitable combination as known by those skilled in the art, as well as any combination of the foregoing configurations. According to one exemplary configuration of the therapy pad holder 90, the pouch 92 can be formed from a netting that can be stretched in one or more directions (e.g., transverse and/or lateral), allowing the pouch 92 to conform to, and securely hold, a variety of therapeutic pad configurations having different sizes and shapes while providing for enhanced thermal transfer therethrough. For example, as shown in FIG. 7, the pouch 92 can conform to the irregularly shaped therapeutic pad 96, which has a reduced width central portion.

The fastener system 98 can be attached to the pouch 92 and can comprise any type of system capable of removably attaching the therapy pad holder 90 to one or more of the garment 16, the sleeve 60, one or more of the sleeves 72, 74, 76, 78 of the compression support system 70, or the therapeutic wrap 102. For example, the fastener system 98 can be a hook and loop fastener system (or one component thereof), such as Velcro®. However, those skilled in the art will understand that any other suitable fastener system can be used for attaching and detaching the therapy pad holder 90 to one or more of the garment 16, the sleeve 60, one or more of the sleeves 72, 74, 76, 78 of the compression support system 70, or the therapeutic wrap 102, such as but not limited to, an adhesive system, a magnetic system, a zipper system, a button system, or a clip system. As shown in FIG. 7, the fastener system 98 can be disposed about a periphery of the exterior of the pouch 92, across the exterior surface of the pouch 92, or a combination thereof, and can have a minimal surface area, thus maximizing the surface area of the pouch 92 exposed to the user and thermal transfer therewith. According to one exemplary configuration of the therapy pad holder 90, the fastener system 98 can comprise a hook and loop material that can be stretched in one or more directions (e.g., lateral and/or transverse), so that the fastening system 98 can stretch and/or move in unison with the pouch 92 and conform to the therapy pad 96.

The therapy pad 96 can be inserted into the pouch 92 via the one or more openings 94. Additionally, the one or more openings 94 can be provided with a closure mechanism (not shown) to aid in securing the therapy pad 96 inside the pouch 92. For example, the closure mechanism can comprise a hook and loop fastener system inside the pouch 92, a flap system, an adhesive system, a zipper system, a button system, a magnetic system, a clip system, or any other suitable system to retain the therapy pad 96 inside the pouch 92. The one or more openings 94 can also provide for connections to the therapy pad 96 from an exterior of the pouch 92. For example, as shown in FIG. 7, the therapy pad 96 can include one or more conduits 100a and 100b extending therefrom. The conduits 100a and 100b can be in communication with a fluid reservoir (not shown) and can be used to circulate fluid (e.g., hot or cold water) through the therapy pad 96. In the case of an electrically powered heating pad, the one or more openings 94 can allow an electrical cord to be passed therethrough.

FIG. 8 is a front view of a therapeutic wrap 102 of the present disclosure. As will be described in greater detail hereinbelow, the therapeutic wrap 102 can be wrapped around an injured body part (e.g., arm, leg, torso, etc.) of a user and one or more of the therapy pack 12 and therapy pack holders 14, 90 can be attached thereto. For example, the therapeutic wrap 102 can be wrapped around a user's limb following a surgical procedure and one or more of the therapy pack 12 and therapy pack holders 14, 90 can be removably attached to the therapeutic wrap 102 in order to apply hot and/or cold therapy thereto and to provide compressive support to the surgically repaired limb during recovery.

The therapeutic wrap 102 can be formed from a plurality of layers, including, but not limited to, an interior layer 104 and an exterior layer 106. As shown, the therapeutic wrap 102 can be configured to have a generally rectangular shape, but can be provided in any other shape and/or length that can be wrapped around a user's body. The interior layer 104 can be formed from a soft-stretch lining material that is configured for comfort against a user's skin. The interior layer 104 can also be configured to exert a compressive force on or around a user's limb (e.g., knee, elbow, calf, etc.), thereby securing the therapeutic wrap 102 against the limb. The exterior layer 106 can be formed from a material that is capable of engaging the securing strips 24 of the therapy pack 12, the fastening system 34 of the therapy pack holder 14, or the fastener system 98 of the therapy pad holder 90, e.g., a loop-type material. In this regard, the exterior layer 106 can be formed from a four-way stretch material, e.g., a material that can be stretched in one or more directions (e.g., lateral and/or transverse) so that one or more of the therapy pack 12, therapy pack holder 14, and therapy pad holder 90 can be removably attached to the exterior layer 106, the exterior layer 106 can stretch and/or move in unison with the interior layer 104, and the therapeutic wrap 102 can exert a compressive force on or around the user's limb. That is, the material of the exterior layer 106 can be compatible with the securing strips 24 of the therapy pack 12, the fastening system 34 of the therapy pack holder 14, and the fastener system 98 of the therapy pad holder 90, such that the securing strips 24, fastening system 34, and fastener system 98 can engage the material of the exterior layer 106 to secure the therapy pack 12, the therapy pack holder 14, or the therapy pad holder 90 to the therapeutic wrap 102.

Alternatively, at least a portion of the exterior layer 106 can be provided with a fastener system configured to removably attach one or more of the therapy pack 12, therapy pack holder 14, and therapy pad holder 90 thereto. For example, those skilled in the art will understand that suitable fastener systems can include, but are not limited to, an adhesive system, a magnetic system, a zipper system, a button system, or a clip system. According to some aspects of the present disclosure, the interior layer 104 and the exterior layer 106 can be sewn together, however any mechanism for securing the layers together can be utilized. According to still further aspects of the present disclosure, the interior layer 104 and the exterior layer 106 can be integrally formed. As such, the therapeutic wrap 102 can be formed from a single layer of self-adhering elastomeric hook and loop material that provides both compressive support, as well as the capability to removably secure one or more therapy pack holders 14, therapy packs 12, and therapy pads 90 to the exterior surface 106 of the therapeutic wrap 102.

A closure system 108 can be provided on the interior layer 104 and can be configured to removably secure the therapeutic wrap 102 around the user's body. For example, the closure system 108 can be one or more strips of hook and loop material disposed about the periphery of the interior layer 104 and the hook and loop material of the closure system 108 can cooperate with the hook and loop material of the exterior layer 106. More specifically, the closure system 108 can comprise “hook-type” material and the exterior layer 106 can comprise “loop-type” material, or vice versa. As such, when the therapeutic wrap 102 is wrapped around the user's body, opposing ends of the therapeutic wrap 102 can overlap and the hook and loop material of the closure system 108 can contact the hook and loop material of the exterior layer 106, thereby securing the therapeutic wrap 102 around the user. Those skilled in the art will understand that any other suitable fastener system 108 can be used for removably securing the therapeutic wrap 102 around the user's body, such as but not limited to, an adhesive system, a magnetic system, a zipper system, a button system, or a clip system.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure.

1. A wearable therapy system, comprising,
a therapy pack;
a therapy pack holder including a body and a first fastener system, the body being configured to removably receive the therapy pack; and
at least one compression sleeve configured to be worn around a user's limb, the at least one compression sleeve comprising an exterior layer formed of a stretchable material and including a second fastener system, and an interior layer configured to exert a compressive force around the user's limb,
wherein the therapy pack holder is removably attachable to the exterior layer in a plurality of positions with the first fastener system engaging the second fastener system to secure the therapy pack in relation to the at least one compression sleeve.

2. The wearable therapy system of claim 1, wherein the therapy pack comprises a pouch filled with a material capable of retaining heat or cold, or generating heat or cold.

3. The wearable therapy system of claim 1, wherein the first fastener system comprises a first component of a hook and loop fastener system, and the second fastener system comprises a second component of the hook and loop fastener system.

4. The wearable therapy system of claim 1, wherein the interior layer is formed from an elastomeric material.

5. The wearable therapy system of claim 1, wherein the stretchable material of the exterior layer is a four-way stretchable material, and the first fastener system is configured to engage the stretchable material to removably attach the therapy pack holder to the at least one compression sleeve.

6. The wearable therapy system of claim 5, wherein the stretchable material is the second fastener system.

7. The wearable therapy system of claim 1, comprising a plurality of compression sleeves configured to be worn around the user's limb, each of the plurality of compression sleeves comprising at least an exterior layer formed of the stretchable material and including the second fastener system and an interior layer configured to exert a compressive force around the user's limb, wherein the first fastener system is configured to engage the second fastener system such that the therapy pack holder is removably attachable to the exterior layer of each of the plurality of compression sleeves in a plurality of positions in relation to each of the plurality of compression sleeves.

8. The wearable therapy system of claim 7, wherein the plurality of compression sleeves are configured to be stacked on one of the user's limbs and form a compression sleeve support system.

9. The wearable therapy system of claim 7, wherein each of the plurality of compression sleeves are configured to be worn around at least one of the user's thigh, knee, calf, ankle, foot, biceps, elbow, forearm, wrist, and hand.

10. The wearable therapy system of claim 9, wherein one or more of the plurality of compression sleeves are provided in a plurality of different sizes.

11. The wearable therapy system of claim 1, wherein the therapy pack is configured to have a fluid circulated therethrough.

12. The wearable therapy system of claim 1, wherein the body of the therapy pack holder is configured to conform to the geometry of the therapy pack and prevent movement thereof.

13. A wearable therapy system, comprising,
a therapy pad configured to have a fluid circulated therethrough;
a therapy pad holder including a pouch with an opening and a first fastener system, the opening configured to removably receive the therapy pad, and the pouch configured to conform to the geometry of the therapy pad and prevent movement thereof; and
a therapy garment configured to be worn about a user's body, the therapy garment comprising a second fastener system,
wherein the therapy pad holder is removably attachable to the therapy garment in a plurality of positions with the first fastener system engaging the second fastener system to secure the therapy pad in relation to the therapy garment.

14. The wearable therapy system of claim 13, wherein the therapy garment is a shirt, a compression garment, a sweatshirt, a jacket, a pair of pants, a pair of shorts, a glove, a shoulder brace, an elbow brace, a wrist brace, a knee brace, an ankle brace, a compression sleeve, or a therapeutic wrap.

15. The wearable therapy system of claim 13, wherein the first fastener system comprises a first component of a hook and loop fastener system, and the second fastener system comprises a second component of the hook and loop fastener system.

16. The wearable therapy system of claim 13, wherein the pouch of the therapy pad holder is formed from a stretchable netting configured to conform to the geometry of the therapy pad.

17. The wearable therapy system of claim 13, wherein the first fastener system is disposed about a periphery of the pouch.

18. A wearable therapy system, comprising,
a therapy pack;
a therapy pack holder including a body and a first fastener system, the body being configured to removably receive the therapy pack; and
a therapeutic wrap configured to be wrapped about at least a portion of a user's body, the therapeutic wrap comprising a second fastener system on a first side thereof and a third fastener system on a second side thereof,
wherein the therapy pack holder is removably attachable to the first side of the therapeutic wrap in a plurality of positions with the first fastener system engaging the second fastener system to secure the therapy pack in relation to the therapeutic wrap, and
wherein the second fastener system on the first side of the therapeutic wrap is removably engageable with the third fastener system on the second side of the therapeutic wrap to removably secure the therapeutic wrap around at least a portion of the user's body.

19. The wearable therapy system of claim 18, wherein the second fastener system is a four-way stretchable material, the first fastener system is configured to engage the stretchable material to removably attach the therapy pack holder to the therapeutic wrap, and the third fastener system of the therapeutic wrap is configured to engage the stretchable material to removably secure the therapeutic wrap around at least a portion of the user's body.

20. The wearable therapy system of claim 19, wherein the therapeutic wrap includes a first layer formed of the stretchable material and a second layer formed of an elastomeric material.
